(19) United States
(12) Patent Application Publication
(10) Pub. No.: US 2024/0041708 A1
(43) Pub. Date: Feb. 8, 2024

(54) DEHYDRATED SOAP AND CLEANER TABLET COMPOSITION

(71) Applicant: HAPPY GO LUCKY LLC, Orlando, FL (US)

(72) Inventors: Chen LIU, Orlando, FL (US); Paul SEPULVEDA, Orlando, FL (US)

(73) Assignee: HAPPY GO LUCKY LLC, Orlando, FL (US)

(21) Appl. No.: 18/490,352

(22) Filed: Oct. 19, 2023

Related U.S. Application Data
(63) Continuation of application No. 17/682,658, filed on Feb. 28, 2022, now abandoned.

(51) Int. Cl. A61K 8/02; A61K 8/44; A61Q 19/10; A61Q 17/00

(52) U.S. Cl. CPC A61K 8/0216; A61K 8/44; A61Q 19/10; A61Q 17/005; A61K 2800/87; A61K 2800/524

(57) ABSTRACT

A soap or cleaner composition including a preservative including iodopropynyl butylcarbamate, a surfactant, a chelator, a water soluble salt, and a polyol. The composition may also include a disinfectant, a humectant, an acid and various additives. The soap or cleaner composition may be formed into a tablet.

### CROSS-REFERENCE TO RELATED APPLICATIONS

This application is a Continuation of copending application Ser. No. 17/682,658, filed on Feb. 28, 2022, which is hereby expressly incorporated by reference into the present application.

### FIELD OF THE INVENTION

The present invention relates to dehydrated soap and cleaner tablet compositions. In particular, the dehydrated soap and cleaner tablet compositions contain iodopropynyl butylcarbamate (IPBC) as a preservative.

### DESCRIPTION OF BACKGROUND ART

Dehydrated soap tablets and cleaner tablets have been developed to offer consumers an alternative product type that limits plastic and packaging waste. This shift in overall consumer interest towards favoring more ecologically sustainable products has been a recent one, and the size of the shift is still relatively small in many older consumer age groups. For these reasons, tablets as a delivery method or product type are relatively new for many consumer products.

Because of the relative immaturity of the field of dehydrated soap tablets and cleaner tablets, existing products have so far been designed to emulate the ingredients of existing liquid products, such as liquid foaming hand soaps.

Within existing traditional non-tablet cosmetics and personal care products, there are a number of common preservatives, which have traditionally been used in the existing products such as parabens, formaldehyde releasers, isothiazolinones, phenoxyethanol, and organic acids.

These products account for the vast majority of all brands and product lines of existing cosmetic and personal care products and they are widely used ingredients among liquid, cream, and gel products. Existing dehydrated tablet recipes do not deviate from these standard ingredients.

Tablet products that are currently on the market make use of organic acids as their main preservative. However, for reasons that will be outlined below, organic acids are insufficient for use in dehydrated soap tablets and cleaner tablets. Significantly better results can be achieved through the use of an alternative formulation using a non-standard preservative.

### SUMMARY OF THE INVENTION

In a first embodiment the present invention is directed to a soap or cleaner composition including a preservative that includes iodopropynyl butylcarbamate, a surfactant, a chelator, a water soluble salt, and a polyol. The iodopropynyl butylcarbamate may be present in an amount ranging from about 1 ppm to about 160 ppm, or in an amount ranging from about 100 ppm to about 160 ppm. The surfactant may be at least one selected from the group consisting of sodium sulfate, sodium dodecyl sulfate (also known as sodium lauryl sulfate), sodium laureth sulfate, sodium lauryl glutamate, sodium glutamate (also known as sodium cocoyl glutamate), disodium cocoyl, glutamate, sodium coco-sulfate, cocamidopropyl betaine, cocamidopropyl hydroxysultaine, sodium cocoyl isethionate, sodium lauroyl methyl isethionate, sodium lauroyl sarcosinate, potassium N-cocoyl glycinate, potassium cocoyl glutamate, hydroxysultaine, sodium xylene sulfonate, and sodium lauryl sulfoacetate. The chelator may be at least one selected from the group consisting of ethylenediaminetetracetic acid disodium, tetrasodium glutamate diacetate, sodium triphosphate, tetrasodium pyrophosphate, and trisodium ethylenediamine disuccinate. The water soluble salt may be at least one selected from the group consisting of sodium carbonate, and sodium bicarbonate. The polyol is at least one selected from the group consisting of ethylene glycol, polyethylene glycol, glycerol and propylene glycol alginate.

The soap or cleaner composition may also include at least one disinfectant. The disinfectant may be selected from the group consisting of trichloroisocyanuric acid and ammonium salts. The soap or cleaner composition may also include at least one humectant selected from the group consisting of xylitol, sorbitol, and hydrogenated starch hydrolysates. The soap or cleaner composition may also include at least one acid selected from the group consisting of citric acid, furmaric acid, salicylic acid, lactic acid, glycolic acid, sodium hyaluronate, sodium hyaluronate crosspolymer, lauric acid, sodium levulinate, gluconic acid, and sulfamic acid. The composition may be formed into a tablet. The composition may be soluble in water.

The soap or cleaner composition may also include an additive for cosmetic compositions. The additive may be selected from the group consisting of stearamidopropyl dimethylamine, histidine, glyceryl stearate citrate, behentrimonium methosulfate, sodium and PPG-26-Buteth-26, glycine, niacinamide, ascorbic acid, ceramide, aspartic acid, alanine, tartaric acid, serine, valine, isoleucine, proline, threonine, phenylalanine, arginine, sodium pyrrolidone carboxylic acid, glutamic: acid, aspartic acid, ferulic acid, palmitic acid, stearic acid, lecithin, glucose, fructose, maltose, dextrin, carbamide, allantoin, chlorphenesin, azelaic acid, polyquaternium caffeine, laureth-23, tocopheryl succinate polyethylene glycol (TSPG), beta carotene, tranexamic acid, alpha-arbutin, mandelic acid, N-acetyl glucosamine, and sodium metasilicate. The soap or cleaner composition may also include an additive for floor cleaner compositions. The additive may be selected from the group consisting of sodium hydroxide, sodium percarbonate, ethoxylated alcohols, potassium permanganate, and urease. The soap or cleaner composition may also include an additive for industrial cleaner compositions. The additive may be selected from the group consisting of sodium hypochlorite and ethoxylated alcohols. The soap or cleaner composition may also include an additive for specialized surface cleaner compositions. The additive may selected from the group consisting of lauramine oxide, bentonite, sodium silicate, and hydroxypropyl methylcellulose.

In a second embodiment the present invention is directed to a rehydratable tablet including the soap or cleaner composition. The rehydratable tablet is a hand soap, face wash, floor cleaner, carpet cleaner, pet stain cleaner, fabric softener, stone cleaner, steel cleaner, odor removing cleaner, or industrial cleaner.

A third embodiment of the present invention relates to a rehydratable surface cleaner tablet including the soap or cleaner composition.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to one of ordinary skill in the art from this detailed description.

### DETAILED DESCRIPTION OF THE INVENTION

The present invention is directed to a dehydrated hand soap and cleaner tablet composition.

The inventors of the present invention tested and pioneered the use of iodopropynyl butylcarbamate (IPBC) as a preservative for dehydrated tablet compositions. Using this method, significant improvements over existing formulas using organic acids as the preservative, achieving typical post-rehydration stability periods exceeding four months and with no test subjects experiencing contaminant growth in less than six months, were obtained. This is in contrast to standard formulations preserved with organic acids, which experience frequent and unpredictable contaminant outbreaks in time periods sometimes as short as two weeks. In fact, in one case numerous separately ordered and individually sealed tablets from a commercially available tablet product line using an organic acid preservative spoiled within their wrapping even prior to any rehydration tests. In general, it is crucial for a retail product to have a shelf stable tablet, and the composition of the invention achieves tablets that should remain stable for an extended period of time measured in months, or even years.

The formulation detailed in this patent includes a mass of IPBC equivalent to a 100 ppm to 160 ppm when the tablet has been fully rehydrated. This concentration remains well within the limits expressed by cosmetic regulations, while simultaneously providing sufficient inhibitory strength to suppress commonly encountered microbes.

### Mechanism of Effect and Preservative Effectiveness

IPBC was first developed in the 1970s. Despite its age, the exact mechanism of IPBC's antimicrobial effect is not well understood. However, based on the relative ineffectiveness of similar compounds lacking iodine, that element appears to play a key role. Though the mechanism is unclear, the effectiveness of IPBC as a preservative, particularly as a fungicide, has been widely documented in numerous studies.

As noted in the Cosmetic Ingredient Review, IPBC is proven to be effective against molds, mildews, yeasts, fungi, algae, and bacteria at varying concentrations. For instance, mold/mildew/yeast/fungi have a minimum inhibitory concentration (MIC) of 10 ppm or less; algae have an MIC of 50 ppm or less; and for bacteria, MICs range from 50 ppm to over 1000 ppm. Publications by commercial providers of IPBC or IPBC-derived fungicides list similar results for their own products.

At the rehydrated concentration level of the present invention, IPBC would be sufficient to severely inhibit or remove virtually any fungi or algae and would have a significant inhibitory effect against most strains of bacteria.

### Operating Environment of the Tablets

There are two key differences between the operating environments of tablet products and their conventional alternatives: (1) Tablet ingredients must be compatible with the process of physically manufacturing tablets; and (2) formulations intended for rehydration face far more potential contaminants that must be addressed. The composition of the present invention which uses IPBC addresses both of these concerns over existing formulas.

The first condition above requires that the preservative must come in a dry or solid form. Phenoxyethanol, for example, cannot be used as a preservative for tablet compositions because it is a liquid. Any moisture in the mix will cause clumping, physically disrupting the machines that produce tablets by either jamming the mold feeding lines or causing buildup within the molds which in turn creates overheating and tablet breakage. Clumping would also prevent even distribution of liquid chemicals during the mixing process.

The limitation on liquid ingredients also has secondary effects. Specifically, ingredients used within the tablet must not react with each other in a manner that releases liquid. Some preservatives that come in solid form like benzalkonium chloride are not appropriate for tablet use because they interact with anionic surfactants and create moisture, causing the aforementioned disruptions within the equipment.

In addition, liquid ingredients are also restricted due to solubility. Many preservatives, like phenoxyethanol, are more soluble in oils than in water (phenoxyethanol is also used in some cases as a solvent for other preservatives). Since oils, as noted above, would create clumping and disrupt the production process, they cannot be included in dehydrated tablet products. Many preservatives are not soluble in water, or are only soluble within narrow ranges. These constraints create further challenges for the use of common preservatives.

The second condition above derives from the unpredictable environment for rehydration. Since liquid manufactured products are mixed and filled into bottles in a very controlled factory environment, their exposure to unknown contaminants is minimized. Even when liquid soap and cleaning products are refilled using multi-bottle refill containers, the only outside material entering the bottle is the sterile liquid mix being poured into the reusable bottle from the larger stock container of the liquid soap or cleaning product. In other words, no unpredictable outside liquids (and their associated microbial contents) are involved.

Tablets, on the other hand, operate under much different circumstances. Though the tablet itself is produced in a sterile environment, from the moment the packaging is opened the tablet composition faces a variety of contamination sources, such as a user's unwashed hands, contaminants contained within the water supply, hard tap water (containing higher than normal alkalinity due to the presence of minerals, which will reduce the effectiveness of some preservatives) and the presence of microbes on the faucet itself due to infrequent cleaning. These contaminant sources present a significant risk of introducing outside contaminants into the soap or cleaning product made from a rehydrated tablet compared to traditional liquid products.

One significant problem with existing soap and cleaning product tablets currently on the market is that the formula for the tablet is designed as if it operates in the same controlled environment as liquid products and the additional contamination risk associated with rehydrated products is not addressed, and therein lies a significant problem with existing formulations that the present invention resolves.

Existing tablet formulations use standard preservatives such as organic acids. The most commonly used preservative for rehydratable soap and cleaning product tablets is sodium benzoate. However, existing formulas that use organic acids as the preservative experience production limitations stemming from the differences in liquid and solid product delivery. The IPBC containing composition of the present invention offers multiple improvements in production.

One consideration regarding compositions of the invention is that the physical size and mass of soap and cleaning product tablets represents a practical design constraint on how any composition must be prepared. Organic acids require a high concentration to be effective. For example, for the fungus Candida albicans, the MIC for IPBC is about 8 ppm, whereas the MIC for sodium benzoate is about 2500 ppm.

Formulas using organic acid preservatives originated with liquid products, where the majority of the product by mass and volume is water and additional ingredients can easily be accommodated by reducing the amount of water in the liquid mixture. Tablets, on the other hand, are constrained by the size limitations of both the rotary press molds and standard commercial bottle size molds. Because tablets are constrained in size for manufacturing purposes, the ability to reduce the amount of preservative used itself represents a significant improvement over currently used preservatives. The reduction in amount of preservative needed represents a substantial reduction in mass and volume taken up by the preservative in the tablet, allowing the savings in mass and volume to be devoted to other ingredients that can enhance the product's appeal (e.g., improved fragrance or hand feel). The reduction in the amount of preservative used also relates to second-order improvements for the formulations. The organic acids most commonly used in soap and cleaning product tablets have an inherent acidity. If more preservative is added, other additional ingredients are required to counterbalance the preservative's effect on properties, such as hand feel. This raises production and packaging costs, and eventually confronts the physical constraints imposed by both the bottles and pumps/sprayers (which have standard neck width sizes) or the molds and other production equipment. It also further reduces the ability to include additional discretionary components to improve, e.g. feel, scent, color etc.

Sodium benzoate has an additional disadvantage of being active as an antimicrobial agent at lower solution pH values. To maximize its preservative effects, compositions need to minimize the pH. This presents an additional formulation challenge for soap-type products where a lower pH is more likely to cause skin irritation. It also makes sodium benzoate less effective in a hard water environment, highlighting another key difference between a controlled water environment (i.e., liquid products produced in a factory setting) versus an uncontrolled water environment (i.e., rehydrated tablets where the user provides the water).

IPBC is significantly more potent on a ppm basis, as well as being non-irritating and pH neutral, thus overcoming the disadvantages and problems associated with currently used preservatives for soap and cleaning product tablets.

Due to its age, IPBC has been extensively studied for many purposes. Multiple studies have reached the following conclusions about IPBC's potential effects: non-persistent in water and soil; no evidence of carcinogenic potential; no sign of bioaccumulation; no detrimental reproductive or developmental effects; unlike other compounds (e.g., parabens) there is no evidence of phototoxicity; noncomedogenic at comparable concentrations; and has a very low rate of contact sensitization. Any reported negative effects were only seen with either (1) a vastly higher concentrations than would be found in any commercial application covered by the present invention or (2) direct inhalation of concentrated IPBC, which would not be an issue with the present invention is a rehydrated liquid solution, rather than e.g. an aerosol.

### Examples

The invention being thus described, one skilled in the art would readily understand that the same may be varied in many ways. Such variations are not to be regarded as a departure from the spirit and scope of the invention, and all such modifications as would be obvious to one skilled in the art are intended to be included within the scope of the following claims.

As part of the development process of the present invention, a variety of different compositions were tested to achieve the present invention. The objective of the present invention is to maximize shelf life and physical characteristics of the soap or cleaner compositions, while minimizing negative effects on either the environment and/or the production process itself.

### Testing Criteria

The key goals for the present invention, given that it is intended to be used for bottles that will be refilled many times to reduce plastic waste, are for the rehydrated formula to:
(a) be stable once rehydrated, meaning that the solution would no discernible microbial growth over a multi-month period of time and would maintain its physical characteristics, and
(b) work within an acceptable manufacturing process (both physically and in terms of cost).

An important feature of the present invention is product that has an improved shelf-life when rehydrated. The tablet of the present invention has a shelf-life upon rehydration of at least 3 months, of at least 4 months, of at least 5 months, of at least 6 months, of at least 9 months, of at least 1 year, of at least 18 months, or of greater than 2 years.

The IPBC used in the invention is used in an amount of 1 ppm to 160 ppm, more preferably 1 ppm to 25 ppm to inhibit fungal growth, more preferably 25 ppm to 50 ppm to inhibit algae growth, and more preferably 50 ppm to 160 ppm to inhibit bacterial growth.¹

In terms of physical characteristics measured, once the test tablets had been rehydrated, several observables were tracked each day such as scent, clarity, presence of particulates and pressure.

Scent was measured by comparing the scent each day to the original scent of an initially rehydrated bottle. When microbial activity is present it can create compounds, which add a distinctly unpleasant odor to the compositions. 2 Given the sensitivity of the human nose to such compounds, this would be a clear aesthetic failure for the product even if overall microbial growth remained below a level that could be harmful or otherwise impact use.

Regarding clarity, initially rehydrated solutions are completely transparent. Any change in clarity indicates uncontrolled microbial growth within the solution.

Once the initial dissolution process is complete, a rehydrated solution should contain no free-floating particulates. The appearance of any particles within the solution was recorded and visually evaluated. There are two possible explanations for particles: 1) crystallization, which can occur to a small degree due to temperature, since temperature affects solubility, and 2) microbial growth. Daily tracking of particle growth can be used to differentiate between the two causes. Crystallization is a one-time event, whereas particles caused by microbial growth will continue to grow in size. Similarly, structured growth, such as the formation of a biofilm from the microbes, would be considered a sign that this test had failed.

Microbial activity can also, in some cases, create gas as a metabolic byproduct. Bottles can be checked daily for internal pressure increase as an early indicator of microbial growth.

### Tablet Formation Process and Use

To make the tablet of the invention, the components of the soap or cleaning product are placed in a dehydration chamber before mixing to remove any remaining moisture that might be present. Once all components have been dried, they are measured and mixed via a rotating powder mixer that continuously stirs the mixture to ensure even distribution of ingredients. The base ingredients are typically added first. Base ingredients include e.g. acids and sodium carbonate, then the active ingredients are added. The fully-blended mixture is then fed into a tablet molding machine. For the following Examples the tablets were molded using a rotary tablet press, as are all industrial-scale tablet products. The tablet machine is a fully-automated production device that consists of a set of rotating hydraulic pistons paired to adjustable tablet molds. As the piston assembly rotates, the dry powder is filled into the mold and the piston then compresses the powder into disc-shaped tablets (though other mold shapes are available). The size of the discs is variable and determined by the sized of the mold setting used. In the examples of the present invention described herein, the tablets were ¾″ in diameter and ⅜″ thick and had a mass 4 g each. After being compressed in the mold, the tablets are then ejected into a collection basket. The unwrapped tablets from the basket are subsequently fed into an automated wrapping machine, which adds individual paper or plastic wrapping to each tablet.

Upon use, the tablet(s) are typically added to water by a user. The tablets have an effervescent reaction, which gradually breaks up the tablet and disperses the ingredients into the water.

The tablets may contain several major categories of ingredients such as preservatives, surfactants, acids, chelators, water soluble salts, polyols, humectants, disinfectants, dyes, scents, and other additives.

Preservatives may be present in the tablets at a percentage of less than <1%. The preservative may include IPBC, sodium benzoate, potassium sorbate, methylparaben, benzisothiazolinone.

Surfactants are the primary cleaning agent in the tablets, and may be present in the tablets at a percentage of from 10% to 30%. The surfactants may include sodium sulfate sodium dodecyl sulfate (also known as sodium lauryl sulfate), sodium laureth sulfate, sodium lauryl glutamate, sodium glutamate (also known as sodium cocoyl glutamate), disodium cocoyl glutamate, sodium coco-sulfate, cocamidopropyl betaine, cocamidopropyl hydroxysultaine, sodium cocoyl isethionate, sodium lauroyl methyl isethionate, sodium lauroyl sarcosinate, potassium N-cocoyl glycinate, potassium cocoyl glutamate, hydroxysultaine, sodium xylene sulfonate, and sodium laurel sulfoacetate.

The tablets may also contain acids, which have a separate functional role from the chelator. The acids control the pH, which is important to maintain max preservative effectiveness and counteract water hardness. The acids also increase shelf-life, and react with sodium bicarbonate to produce CO₂ and create the effervescent reaction. In skincare, acids may act as a cleanser or a mild exfoliant. In cleaning products, the acids are a mild cleaner that are able to clean soap scum or mineral deposits. The acids may also function as a chelating agent. The acids may include citric acid, fumaric acid, salicylic acid, lactic acid, glycolic acid, sodium hyaluronate, sodium hyaluronate crosspolymer, Lurie acid, sodium levulinate, gluconic acid, and sulfamic acid.

The tablets may also contain at least one chelator, which may be present in the tablet at a percentage of less than <5%. The chelator binds with metal ions to prevent them from reacting with the surfactant and disrupting the cleaning action. EDTA specifically also has a synergistic effect with many preservatives by causing a disruption in the outer membrane of cells (particularly Gram-negative bacteria) that makes them more permeable to other preservatives. The chelator may include EDTA-2NA, tetrasodium glutamate diacetate, sodium triphosphate, tetrasodium pyrophosphate, and trisodium ethylenediamine disuccinate.

The tablets may also contain water soluble salts, which may be present in the tablet at a percentage of from 20% to 40%. Water soluble salts act as a mild desiccant that absorbs any moisture during the mixing process and reacts with the acid in water to create an effervescent effect. The water soluble salts may include sodium carbonate and sodium bicarbonate.

The tablets may also contain polyols, which may be present in the tablet at a percentage of less than 10%. The polyols may include ethylene glycol, polyethylene glycol, glycerol, and propylene glycol alginate.

The tablets may also include a humectant for skincare, which may be present in the tablet at a percentage of less than 10%. The humectant may include xylitol, sorbitol, and hydrogenated starch hydrolysates. When xylitol acts as a humectant, it moisturizes and improves the skin. It also has a mild preservative effect, inhibiting the growth of bacteria.

The tablets may also include disinfectants which may be present in the tablet at a percentage of less than <5%. The disinfectant may include trichloroisocyranuric acid (TCCA) and ammonium salts, such as benzalkonium chloride.

The tablets may also include ingredients for aesthetic purposes, which may be present in the tablet at a percentage of less than 5%. The ingredients for aesthetic components may include a variety of dyes that are approved by the Food and Drug Administration for cosmetic color additives, and dehydrated scent additives to add a desired fragrance.

In addition to an all-purpose/glass/bathroom cleaning composition, other cleaners such as floor cleaner, carpet cleaner, pet stain cleaner, fabric softener/detergent, stone/steel cleaner, odor removing cleaner, and industrial cleaner may also be formulated. The formulations may include substitutions of the specialized ingredients for the application. The tablets may include other additives such as sodium hydroxide (carpet cleaner/stain remover), sodium hypochlorite (industrial cleaner), sodium percarbonate (carpet cleaner/stain remover), ethoxylated alcohols (industrial cleaner, carpet cleaner, stain remover), potassium permanganate (pet stain remover), urease (pet stain remover), lauramine oxide (stone/steel cleaner), bentonite (fabric softener/detergent); sodium silicate (fabric softener/detergent) hydroxypropyl methylcellulose (wood cleaner), polyacrylic acid (also known as carbomer, for thickening applications), polyacrylate crosspolymer-6, hydroxyethyl cellulose (for thickening applications), xantham gum (for thickening applications), gellan gum (for thickening applications), magnesium aluminum silicate (for thickening applications), stearamidopropyl dimethylamine (hair care); histidine (cosmetic antioxidant); glyceryl stearate citrate (cosmetic emollient); behentrimonium methosulfate (cosmetic emollient); sodium and PPG-26-Buteth-26 (cosmetic hydrating), glycine (cosmetic conditioning), niacinamide (cosmetic), ascorbic acid (cosmetic), ceramide (cosmetic), aspartic acid (cosmetic); alanine (cosmetic); tartaric acid (cosmetic), serine (cosmetic), valine (cosmetic), isoleucine (cosmetic), proline (cosmetic), threonine (cosmetic), phenylalanine (cosmetic), arginine (cosmetic), sodium pyrrolidone carboxylic acid (also known as PCA, cosmetic), glutamic acid (cosmetic), aspartic acid (cosmetic), ferulic acid (cosmetic), palmitic acid (cosmetic), stearic acid (cosmetic), lecithin, glucose (cosmetic), fructose (cosmetic), maltose (cosmetic), dextrin (cosmetic), carbamide also known as urea. (cosmetic), allantoin (cosmetic), chlorphenesin, azelaic acid, polyquaternium (cosmetic—hair), caffeine, laureth-23, tocopheryl succinate polyethylene glycol (TSPG), beta carotene, tranexamic acid, alpha-arbutin, mandelic acid, N-acetyl glucosamine, and sodium metasilicate.

### Testing Process

In the inventive examples and the comparative examples described herein, each bottle tested was rehydrated using the following process to ensure consistency across all sample results.

First, rehydration was performed within a fresh, clean bottle of the appropriate volume which is 500 mL for hand soap testing, and 1 L for cleaner testing.

Next, when inserting the tablets into the bottle for rehydration, each tablet was removed from the plastic protective coating, then handled for several seconds by the testing individual's fingertips. As the testing is meant to measure the ability of the preservative to prevention microbial growth, the rehydration steps were done to create a typical “real-world” environment, in which the person using the tablet rehydrates it with the possibility of introducing environmental microbes. The tablets in all samples of a test set were handled similarly and during the same span of time to maintain a similar microbial transfer opportunity.

Next, rehydration for all bottles in a test group was performed at the same time, using water from the same source for each test session. This was done to maintain as much consistency as possible between test bottles and from one test group to the next. No cleaner was applied to the faucet prior to rehydration, since the test scenario is meant to recreate the possible introduction of outside microbes as would be typical when the tablet is being used by a consumer.

Once rehydrated, bottles were stored in a cool, dimly lit interior room to simulate the common conditions for a bathroom. Temperature and relative humidity during production and storage are maintained between 20° C. to 24° C. and 40% to 60%, respectively, to prevent microbial growth and/or contamination and to prevent clumping during production.

The bottles with rehydrated tablets were examined daily to record the properties of scent, clarity, particular matter and pressure. Samples were evaluated on a pass/fail basis. Any change from the base value on any of the four key criteria was recorded as a failure for the formulation. The day count of the failure relative to the start date was recorded, with the day of reconstitution being considered as Day 1.

In Examples 1 and 2, the concentration of IPBC was in the 100 ppm to 160 ppm range. This range inhibits the growth of all three categories of microbes. An amount of 100 ppm is a minimum safe amount to inhibit most types of microbial growth. IPBC in the range of 100 ppm to 160 ppm significantly inhibits the growth of fungus and yeast. IPBC is less effective against bacteria in relative terms at this amount but in absolute terms by weight it is still quite effective against bacteria as well. 100 ppm is the concentration where IPBC had an inhibitory effect against most bacteria tested against, and therefore determined the minimum IPBC used should be approximately 100 ppm.

The percentages in the Tables below are per individual tablet. The ppm values are for the fully rehydrated solution, which in the case of the hand soap was three 4 g tablets per 500 mL and for the cleaner was one 4 g tablet per 1 L.

### Test Results

The following tables document compositions and the results of some samples of the test results. A “P” value in the Tables indicates that the sample passed according to the evaluation criteria. An “F” values in the Tables indicates that this was the failure point for this sample. The failure was recorded based on the sample status at the time of failure according to one or more metrics. In other words, the samples may have continued on to fail on other metrics. For example, a failure marked for scent may have also developed visible biofilm structures over time, but these had not yet appeared as of the initial failure date. The numeric value represents the number of days from the start of the test when failure occurred, such as “D 10” indicates a failure in that observable on the tenth day of observation.

### Example 1: Tablet Composition for a Cleaning Formula

The composition for the cleaning formulation is shown in Table 1. Four separate trials, 9 (CL) to 12 (CL), were conducted for the composition as shown in Table 3.

TABLE 1

| Chemical Name | Percentage (%) per Tablet |
|---|---|
| Citric acid | 30.0 |
| Sodium carbonate | 20.0 |
| Fumaric acid | 20.0 |
| Sodium sulfate | 10.0 |
| Sodium benzoate | 7.5 |
| Iodoproponyl butylcarbamate | 2.5 |
| Polyethylene glycol | 2.0 |
| Ethylenediaminetetracetic acid | 2.0 |
| disodium (EDTA-2Na) | |
| Trichloroisocyanuric acid | 0.4 |
| Perfume | 3.0 |
| Dye | 2.5 |

### Example 2: Tablet Composition for a Hand Soap Formula

The composition for the hand soap formulation is shown in Table 2. The formula was designed for 500 mL of water, with 3 tablets. Eight separate trials, 1 (HS) to 8 (HS), were conducted for this formula as shown in Table 3.

TABLE 2

| Chemical formula | Percentage (%) per tablet |
|---|---|
| Citric acid monohydrate | 28.0 |
| Sodium carbonate | 25.0 |
| Sodium bicarbonate | 15.0 |
| Sodium dodecyl sulfate | 14.0 |
| Ethylene glycol | 5.0 |
| Glycerol | 5.0 |
| Xylitol | 5.0 |
| Iodopropynyl butylcarbamate | 0.5 |
| Ethylenediaminetetracetic acid | 0.4 |
| disodium (EDTA-2Na) | |
| Perfume | Variable |
| Dye | Variable |

The results for Examples 1 and 2 of the present invention can be found in Table 3 below.

TABLE 3

| Sample ID # | Scent | Clarity | Particulates | Pressure |
|---|---|---|---|---|
| 1 (HS) | P | P | P | P |
| 2 (HS) | P | P | P | P |
| 3 (HS) | P | P | P | P |
| 4 (HS) | P | P | P | P |
| 5 (HS) | P | P | P | P |
| 6 (HS) | P | P | P | P |
| 7 (HS) | P | P | P | P |
| 8 (HS) | P | P | P | P |
| 9 (CL) | P | P | P | P |
| 10 (CL) | P | P | P | P |
| 11 (CL) | P | P | P | P |
| 12 (CL) | P | P | P | P |

*HS represents a hand soap formulation according to Table 2.
*CL represents a cleaner formulation according to Table 1.

### Example 3: Tablet Composition for a Face Wash Formula

A composition for a face wash formulation is shown in Table 4.

TABLE 4

| Chemical Name | Percentage (%) per tablet |
|---|---|
| Citric acid monohydrate | 25 |
| Sodium carbonate | 24 |
| Sodium bicarbonate | 15 |
| Sodium Lauroyl Methyl Isethionate | 10 |
| Sodium Xylene Sulfonate | 5 |
| Ethylene glycol | 5 |
| Glycerol | 5 |
| Xylitol | 5 |
| Histidine | 3 |
| Iodopropynyl butylcarbamate | 0.5 |
| Ethylenediaminetetracetic acid | 0.4 |
| disodium (EDTA-2Na) | |
| Perfume | Variable |
| Dye | Variable |

### Example 4: Tablet Composition for a Carpet Stain Remover Formula

A composition for a carpet stain remover formulation is shown in Table 5

TABLE 5

| Chemical Name | Percentage (%) per Tablet |
|---|---|
| Sodium Percarbonate | 87 |
| Sodium Lauryl Sulfate | 10 |
| Iodopropynyl Butylcarbamate | 0.8 |
| EDTA-2NA | 0.7 |
| Perfume | Variable |

### Example 5: Tablet Composition for an Industrial Cleaner Formula

A composition for an industrial cleaner formulation is shown in Table 6.

TABLE 6

| Chemical Name | Percentage (%) per Tablet |
|---|---|
| Ethoxylated Alcohols | 30 |
| Sodium carbonate | 20 |
| Citric acid | 20 |
| Fumaric acid | 20 |
| Polyethylene glycol | 2 |
| Ethylenediaminetetracetic acid | |
| disodium (EDTA-2Na) | 2 |
| Iodoproponyl butylcarbamate | 1.25 |
| Perfume | Variable |
| Dye | Variable |

### Example 6: Tablet Composition for a Steel/Granite Cleaner

A composition for a steel/granite cleaner formulation is shown in Table 7.

TABLE 7

| Chemical Name | Percentage (% ) per Tablet |
|---|---|
| Citric acid | 30.0 |
| Sodium carbonate | 20.0 |
| Fumaric acid | 17.9 |
| Sodium sulfate | 10.0 |
| Lauramine Oxide | 10.0 |
| Iodoproponyl butylcarbamate | 2.5 |
| Polyethylene glycol | 2.0 |
| Ethylenediaminetetracetic acid disodium | 2.0 |
| (EDTA-2Na) | |
| Perfume | 3.0 |
| Dye | 2.5 |

### Comparative Example 1: Tablet Composition for a Hand Soap Formula

The composition for the comparative hand soap formula is shown in Table 8. The results of the comparative hand soap composition are shown in Table 9.

TABLE 8
Chemical Name
Sodium lauryl sulfate
Sodium lauryl glutamate
Sodium C14-16 olefin sulfonate
Sodium bicarbonate
Citric acid anhydrous
PEG6000
Sodium benzoate
Fragrance
EDTA-2NA

TABLE 9

| Sample ID # | Scent | Clarity | Particulates | Pressure |
|---|---|---|---|---|
| 1 | P | P | P | P |
| 2 | F | F | P | P |
| | (D10) | (D10) | | |
| 3 | F | F | P | P |
| | (D12) | (D12) | | |
| 4 | P | P | P | P |
| 5 | P | P | P | P |
| 6 | P | P | P | P |

The hand soap of Comparative Example 1 experienced a severe failure in about 30% of trials within 4 weeks.

### Comparative Example 2: Tablet Composition for a Hand Soap Formula

The composition for the comparative hand soap formula is shown in Table 10. The results of the comparative hand soap composition are shown in Table 11.

TABLE 10
Chemical Name
Sodium hydrogen carbonate
Citric acid
Sodium carbonate
Polyethylene adipate
Sodium dodecyl sulfate
Multi-enzyme

TABLE 11

| Sample ID # | Scent | Clarity | Particulates | Pressure |
|---|---|---|---|---|
| 1 | P | P | F | P |
| | | | (D22) | |
| 2 | P | P | F | P |
| | | | (D18) | |
| 3 | P | P | F | P |
| | | | (D24) | |

This soap formula experienced a severe failure in 100% of the trials within 4 weeks. In addition, the user experience (primarily scent and hand feel) were poor for this formula, so only a handful of tests were performed.

### Comparative Example 3: Hand Soap Formula (for 500 mL with 3 Tablets)

TABLE 12

| Chemical Name | Percentage (%) |
|---|---|
| Citric acid | 35.0 |
| Sodium bicarbonate | 25.0 |
| Alkyl polyglycoside | 25.0 |
| Polyethylene glycol (PEG) | 5.0 |
| Benzalkonium chloride (BKC) | 0.1 |
| Fragrance | 7.0 |

### Comparative Example 4: Cleaner Formula (for 1 L)

TABLE 13

| Chemical Name | Percentage (%) |
|---|---|
| Citric acid | 40.0 |
| Sodium bicarbonate | 20.0 |
| Alkyl polygylcoside | 20.0 |
| Polyethylene glycol (PEG) | 5.0 |
| Benzalkonium chloride (BKC) | 0.1 |
| Fragrance | 10.0 |
| Dye | 5.0 |

Comparative Examples 3 and 4 use benzalkonium chloride (BKC) as an alternative preservative.

Although BKC was somewhat effective in stability testing, it failed the requirement for production processing as substantial production issues were encountered. In this regard, BKC reacted with surfactants in the mixture, which resulted in moisture. The presence of moisture disrupted the production machinery by causing sticking, which led to the machine overheating and tablets fracturing during the molding process. These issues resulted in frequent stoppages to clear the machine, along with large amounts of raw materials lost as waste due to the number of broken tablets that would not be sellable and had to be discarded.

The results of testing using Comparative Example 3 are shown in Table 14 as 1 (HS) to 11 (HS). The results of testing using Comparative Example 4 are shown in Table 14 as 12(CL) to 16(CL).

TABLE 14

| Sample ID # | Scent | Clarity | Particulates | Pressure |
|---|---|---|---|---|
| 1 (HS) | P | P | P | P |
| 2 (HS) | P | P | P | P |
| 3 (HS) | P | P | P | P |
| 4 (HS) | P | P | F | P |
| | | | (D14) | |
| 5 (HS) | P | P | P | F |
| | | | | (D7) |
| 6 (HS) | P | P | P | P |
| 7 (HS) | P | P | P | P |
| 8 (HS) | P | P | P | P |
| 9 (HS) | P | P | F | F |
| | | | (D12) | (D12) |
| 10 (HS) | P | P | F | P |
| | | | (D17) | |
| 11 (HS) | P | P | P | P |
| 12 (CL) | F | P | F | P |
| | (D8) | | (D8) | |
| 13 (CL) | P | P | P | P |
| 14 (CL) | P | P | P | P |
| 15 (CL) | P | P | P | P |
| 16 (CL) | P | P | P | P |

Conclusions from Testing

As noted previously, rehydrated soap and cleaner tablets operate in a different environment from traditional liquid soap/cleaner products. Traditional liquid soap/cleaner products are mixed and filled in sterile factory environments, where all ingredients, particularly the water, are specifically kept free of contaminants and the container will remain sterile until opened by the consumer. It is relevant to note that when this sterile manufacturing environment is violated, it can result in a product recall of the affected lots. This indicates an important point about the relative strength of preservatives included in standard liquid manufactured hand soap and cleaner compositions: the preservatives are sufficient to maintain the preservation of an initially sterile solution, but not one with incidental contaminants. Soap and cleaner tablet compositions currently on the market have been modeled after liquid ones, using the same preservatives and, as a result share the same weakness: they are vulnerable to outside contaminants. The testing results confirm this problem with current soap and cleaner tablets: the traditional preservatives used are not sufficient to maintain the shelf life of a soap or cleaner from a rehydrated tablet because the rehydration by the consumer is usually not done in a sterile environment with contaminant-free water.

The present invention overcomes the issues associated with currently available rehydratable soap and cleaner products and IPBC has proven to be highly effective as a tablet preservative agent. Beyond the four-week window used for the testing above, each IPBC-containing test bottle of soap or cleaner remained stable and microbe free for at least four months.

### Manufacturing Process Testing

In addition to the stability performance of the rehydrated mixture, a key part of the testing process was how the formula performed during the manufacturing process. For example, while BKC provided better preservation than other non-IPBC-containing compositions tested, there were significant difficulties associated with BKC during the manufacturing process as a result of chemical interactions with other ingredients.

This highlights another important advantage when comparing between liquid and tablet products containing IPBC: is the IPBC (with regard to other ingredients in the mix) is chemically non-reactive and therefore represents a significant advancement over other previously used preservatives as it satisfies the criteria (a) and (b) listed above.

1. A soap or cleaner composition, comprising:
a preservative comprising iodopropynyl butylcarbamate;
a surfactant;
a chelator;
a water soluble salt; and
a polyol,
wherein the composition is formed into an effervescent tablet, and
wherein the composition is soluble in tap water.

2. The composition of claim 1, wherein the iodopropynyl butylcarbamate is present in an amount ranging from about 1 ppm to about 160 ppm.

3. The composition of claim 2, wherein the iodopropynyl butylcarbamate is present in an amount ranging from about 100 ppm to about 160 ppm.

4. The composition of claim 1, wherein the surfactant is at least one selected from the group consisting of sodium sulfate, sodium dodecyl sulfate (also known as sodium lauryl sulfate), sodium laureth sulfate, sodium lauryl glutamate, sodium glutamate (also known as sodium cocoyl glutamate), disodium cocoyl glutamate, sodium coco-sulfate, cocamidopropyl betaine, cocamidopropyl hydroxysultaine, sodium cocoyl isethionate, sodium lauroyl methyl isethionate, sodium lauroyl sarcosinate, potassium N-cocoyl glycinate, potassium cocoyl glutamate, hydroxysultaine, sodium xylene sulfonate, and sodium lauryl sulfoacetate.

5. The composition of claim 1, wherein the chelator is at least one selected from the group consisting of ethylenediaminetetracetic acid disodium, tetrasodium glutamate diacetate, sodium triphosphate, tetrasodium pyrophosphate, and trisodium ethylenediamine disuccinate.

6. The composition of claim 1, wherein the water soluble salt is at least one selected from the group consisting of sodium carbonate, and sodium bicarbonate.

7. The composition of claim 1, wherein the polyol is at least one selected from the group consisting of ethylene glycol, polyethylene glycol, glycerol, and propylene glycol alginate.

8. The composition of claim 1, further comprising at least one disinfectant.

9. The composition of claim 8, wherein the disinfectant is selected from the group consisting of trichloroisocyanuric acid and ammonium salts.

10. A rehydratable tablet comprising the composition of claim 1.

11. The rehydratable tablet of claim 10, wherein the rehydratable tablet is a hand soap, face wash, floor cleaner, carpet cleaner, pet stain cleaner, fabric softener, stone cleaner, steel cleaner, odor removing cleaner, or industrial cleaner.

12. The composition of claim 1, further comprising at least one humectant selected from the group consisting of xylitol, sorbitol, and hydrogenated starch hydrolysates.

13. The composition of claim 1, further comprising at least one acid selected from the group consisting of citric acid, fumaric acid, salicylic acid, lactic acid, glycolic acid, sodium hyaluronate, sodium hyaluronate crosspolymer, lauric acid, sodium levulinate, gluconic acid, and sulfamic acid.

14. The composition of claim 1, further comprising an additive selected from the group consisting of stearamidopropyl dimethylamine, histidine, glyceryl stearate citrate, behentrimonium methosulfate, sodium and PPG-26-Buteth-26, glycine, niacinamide, ascorbic acid, ceramide, aspartic acid, alanine, tartaric acid, serine, valine, isoleucine, proline, threonine, phenylalanine, arginine, sodium pyrrolidone carboxylic acid, glutamic acid, aspartic acid, ferulic acid, palmitic acid, stearic acid, lecithin, glucose, fructose, maltose, dextrin, carbamide, allantoin, chlorphenesin, azelaic acid, polyquaternium caffeine, laureth-23, tocopheryl succinate polyethylene glycol (TSPG), beta carotene, tranexamic acid, alpha-arbutin, mandelic acid, N-acetyl glucosamine, and sodium metasilicate.

15. The composition of claim 1, further comprising an additive selected from the group consisting of sodium hydroxide, sodium percarbonate, ethoxylated alcohols, potassium permanganate, and urease.

16. The composition of claim 1, further comprising an additive selected from the group consisting of sodium hypochlorite and ethoxylated alcohols.

17. The composition of claim 1, further comprising an additive selected from the group consisting of lauramine oxide, bentonite, sodium silicate, and hydroxypropyl methylcellulose.
